(19) United States
(12) Patent Application Publication
(10) Pub. No.: US 2017/0252520 A1
(43) Pub. Date: Sep. 7, 2017

(54) PUNCTURE NEEDLE

(71) Applicants: NIPRO CORPORATION, Osaka (JP); Food and Drug Administration, Silver Spring, MD (US)

(72) Inventors: Yoshio Higaki, Osaka (JP); Oleg Vesnovsky, Baltimore, MD (US)

(73) Assignees: NIPRO CORPORATION, OSAKA (JP); Food and Drug Administration, Silver Spring, MD (US)

(21) Appl. No.: 15/058,628

(22) Filed: Mar. 2, 2016

(51) Int. Cl. A61M 5/32; A61B 17/34

(52) U.S. Cl. CPC A61M 5/3286; A61B 17/3417; A61M 2205/195; A61B 2017/3454

(57) ABSTRACT

The present disclosure relates, according to some embodiments, to a puncture needle that can prevent the occurrence of coring. A puncture needle according to the present disclosure includes a cannula provided with a lumen. The cannula has a blade surface. The blade surface has: a sharp region located on a tip side of the blade surface and provided with a sharp edge on inner and outer circumferences of the blade surface; and a blunt region located on a base end side of the blade surface and provided with a blunt edge on the inner and outer circumferences of the blade surface. A tip of the blade surface is provided with a thick portion formed by a boundary between two surfaces, and the boundary forms an edge.

### FIELD OF THE DISCLOSURE

The present disclosure relates, in some embodiments, to a puncture needle.

### BACKGROUND OF THE DISCLOSURE

Puncture needles may be configured as disclosed in, for example, Japanese Patent Laying-Open No. 2005-95571. However, a need has arisen for puncture needles with improved properties and/or performance.

### SUMMARY

Existing puncture needles may not effectively prevent coring. Accordingly, a need has arisen for an improved puncture needle. The present disclosure relates, in some embodiments, to a solution for the aforementioned problem. The present disclosure provides, in some embodiments, a puncture needle that may reduce or prevent coring.

A puncture needle, according to the present disclosure, may comprise a cannula provided with a lumen. The cannula has a blade surface, the blade surface has: a sharp region located on a tip side of the blade surface and provided with a sharp edge on inner and outer circumferences of the blade surface; and a blunt region located on a base end side of the blade surface and provided with a blunt edge on the inner and outer circumferences of the blade surface, and a tip of the blade surface is provided with a thick portion formed by a boundary between two surfaces, and the boundary forms an edge.

A puncture needle may provide access to a space through or across a barrier. However, coring may occur when insertion of a needle results in a portion of a barrier (e.g., a disc or plug of barrier material) being released. A released portion of a barrier may occlude and/or obstruct a needle and/or a line to which it may be connected. Accordingly, a need has arisen for annular tools (e.g., puncture needles) with reduced coring.

The present disclosure relates, according to some embodiments, to systems, apparatuses, and/or methods for accessing a space (e.g., a lumen) using an annular tool (e.g., a needle) with reduced (e.g., without or substantially without) barrier material being released in the cavity defined by the annulus of the tool (e.g., within the cavity defined by the wall of the needle).

For example, the present disclosure relates in some embodiments to a puncture needle comprising a cannula provided with a lumen and the cannula having a blade surface. In some embodiments, a blade surface may have (a) a sharp region (i) located on a tip side of said blade surface and (ii) provided with a sharp edge on inner and outer circumferences of the blade surface, and/or (b) a blunt region (i) located on a base end side of said blade surface and (ii) provided with a blunt edge on the inner and outer circumferences of said blade surface. According to some embodiments, a tip of a blade surface may be provided with a thick portion formed by a boundary between two surfaces, where the boundary forms an edge. A blunt region, in some embodiments, may be formed to extend over a half or more of a height of the cannula. In some embodiments, a blade surface may have a first surface located on a base end side and a second surface located on a tip side, and a side surface ridge line which is a boundary between a first surface and a second surface may be located in the blunt region. According to some embodiments, a length of a sharp region may be less than or equal to 40% of a length of a blade surface. A blade surface of a second surface may have a shape having a curvature that becomes gradually smaller from the tip side toward the base end side. In some embodiments a second surface may have a right-side second surface and a left-side second surface, and an angle formed by the right-side second surface with respect to the left-side second surface may be greater than or equal to 66° and less than or equal to 114°. A ratio of a length of a second surface to a length of a blade surface may be greater than or equal to 35% and less than or equal to 50%, according to some embodiments. A sharp edge may be formed on an inner and an outer circumference of a blade surface in a range where a length from a tip of the blade surface is greater than or equal to 30% and less than or equal to 40% of the length of the blade surface, in some embodiments. In some embodiments, an angle formed by a right-side second surface with respect to a left-side second surface may be: (a) greater than or equal to 66° and less than or equal to 94° or (b) greater than or equal to 66° and less than or equal to 74°. A length of a second surface in some embodiments may be greater than or equal to 46% and less than or equal to 54% of a length of a blade surface. In a range where a length from a tip of a blade surface is greater than or equal to 26% and less than or equal to 34% of the length of the blade surface, a sharp edge may be formed on the inner and outer circumferences of the blade surface. In some embodiments a blunt region is formed by blast treatment, and an area of a surface subjected to blast treatment may be larger on a base end side than on a tip side in a first surface, and larger on the base end side than on the tip side in a second surface. According to some embodiments a cannula may be exposed from a hub having a linear shape.

Further the present disclosure relates, in some embodiments, to a puncture needle comprising a needle wall defining a central longitudinal axis, the central longitudinal axis positioned within a first plane and within a second plane that perpendicularly intersects the first plane. A needle wall may comprise: an inner needle wall surface; an outer needle wall surface; a first zone with a circumferentially contiguous tubular configuration that fully encircles a central longitudinal axis; a second zone that partially encircles the central longitudinal axis; and a third zone that only partially encircles the central longitudinal axis and forms a needle tip, the third zone adjoining and extending contiguously from the second phase of the second zone.

According to some embodiments, a second zone may comprise (a) a first phase that adjoins and extends contiguously from the first zone, (b) a second phase that adjoins and extends contiguously from the first phase, and (c) a lateral opening spanning the first and second phases, defining a blade surface consisting of a first portion positioned in the first phase and a second portion positioned in the second phase, and defining an inner blade circumference and an outer blade circumference. In some embodiments, a first portion of a blade surface may be positioned in a third plane with the third plane being oblique to a central longitudinal axis, oblique to a first plane, and perpendicular to a second plane. A second portion of a blade surface may be positioned generally in a plane curve, the plane curve oblique to a central longitudinal axis, oblique to a first plane, perpendicular to a second plane, and oblique to a third plane. In some embodiments a section of a needle tip, perpendicular to a longitudinal axis and taken anywhere in a third zone, may form a partial Reuleaux triangle in which a base is a circular segment of radius (Rx) defined by an outer surface and two straight sides, each defined by a blade surface, and together defining an angle α^{tip} with a length of the two straight sides and a circumference of the circular segment generally decreasing in successive sections perpendicular to a central longitudinal axis moving toward a distal extent of the needle tip. In some embodiments a first portion of a blade surface may be roughened.

In some embodiments of the disclosure, an angle α^{tip} may be from about 70° to about 110°. An angle α^{tip} may be constant across the third zone. According to some embodiments, a section of a second phase of a second zone of a puncture needle may be perpendicular to a longitudinal axis and taken anywhere in the second phase of the second zone may form an annular sector defined by an outer needle wall surface sector, an inner needle wall surface sector, a left blade surface, and a right blade surface, lines extending from the left and right blade surfaces intersecting to form an angle θ. An angle θ may be from about 70° to about 110°, according to some embodiments. In some embodiments an angle θ may be constant across a third zone.

Some embodiments of the present disclosure describe a puncture needle comprising a cannula provided with a lumen, the cannula comprising a puncture portion, and the puncture portion comprising: (a) a first surface, across left- and right-sides, distal to a tip of the puncture portion; (b) a left-side second surface proximal to the tip of the puncture portion; and (c) a right-side second surface proximal to the tip of the puncture portion. In some embodiments, a first surface may connect to a left-side second surface via a left ridge and may connect to the right-side second surface via a right ridge. A left-side second surface and the right-side second surface may intersect at a sharp edge extending from a tip of a puncture portion to a tip-end of a lumen 3, according to some embodiments. In some embodiments, both inner and outer circumferences of a left-side second surface are recessed and curved, and both inner and outer circumferences of the right-side second surface are recessed and curved, from the perspective of a plane defined by the sharp edge and a longitudinal direction of the cannula. In some embodiments a length of a first surface along a longitudinal direction may be between about 46% and about 54% of a length of a puncture portion along the longitudinal direction.

According to some embodiments a first surface may be blast-treated for reducing coring, and part of a left-side second surface and of a right-side second surface that extends from a tip may not be blast-treated. In some embodiments, 70% of a puncture portion, by length along the longitudinal direction, may be blast-treated with the blast-treated part being distal to the tip.

In some embodiments of the disclosure an inclination angle of a sharp edge (α), with respect to a longitudinal direction, may be: (a) greater than an inclination angle of a first surface (β) or (b) greater than an inclination angle of the inner circumference of the left-side second surface, at the tip-end of the lumen, with respect to the longitudinal direction. In some embodiments an inclination angle of a first surface (β) may be smaller than an inclination angle of the inner circumference of a left-side second surface at a left ridge, with respect to a longitudinal direction. In some embodiments, from the perspective of a cross-sectional plane perpendicular to a longitudinal direction, a left-side second surface may define a first line, a right-side second surface may define a second line, and an angle between the first and second lines may be no less than about 70° and no greater than about 110°.

Further, the present disclosure relates, in some embodiments, to a puncture needle comprising a cannula, with the cannula comprising a puncture portion for insertion into a septum. A puncture portion may comprise a blade surface inclined with respect to a longitudinal direction of the cannula, the blade surface comprising: (a) a first surface proximal to a base-end of the blade surface; (b) a left-side second surface proximal to a tip of the blade surface; and (c) a right-side second surface parallel to the left-side second surface with respect to the longitudinal direction of the cannula. In some embodiments part of a blade surface distal to a tip of a puncture portion is blast-treated, and part of the blade surface extending from the tip of the puncture portion is not blast-treated. A first surface may intersect both a left-side second surface and a right-side second surface with at least one common angle less than 180°. According to some embodiments, at least part of a first surface may be blast-treated for reducing coring and parts of a left-second surface and a right-side second surface extending from the tip of the blade surface may not be blast-treated. The present disclosure further relates, according to some embodiments, to a puncture needle comprising a cannula provided with a lumen, the cannula comprising a blade surface with an inner edge circumference and an outer circumference. According to some embodiments, a blade surface may comprise: (a) a blunt region located on a base-end side of the blade surface and (b) a sharp region located on a tip side of the blade surface. A blunt region may comprise, in some embodiments, (i) a first blunt edge on an inner edge circumference of the blade surface and (ii) a second blunt edge on an outer circumference of the blade surface. A sharp region may comprise (i) a first sharp edge on an inner edge circumference of the blade surface and (ii) a second sharp edge on an outer circumference of the blade surface.

According to some embodiments, a sharp region may further comprise a left-side surface and a right-side surface with the left-side surface and the right-side surface intersecting with an angle between about 66° to about 114°, at a straight boundary line extending from a tip end of an outer circumference to a tip end of an inner edge circumference. An inclination angle of a straight boundary line, with respect to a longitudinal direction of a cannula, may be greater than (1) an inclination angle of an outer circumference at a tip end of the outer circumference and (2) an inclination angle of an inner edge circumference at the tip end of the inner edge circumference, according to some embodiments. In some embodiments, a left-side surface and a right-side surface may have a recessed, curved profile in a side view that is parallel to a plane defined by a straight boundary line and the longitudinal direction of the cannula. According to some embodiments, a blunt region may intersect a left-side surface at a first ridge and may intersect a right-side second surface at a second ridge. A length of a sharp region may be between about 46% and about 54% of a length of a blade surface along a longitudinal direction of a cannula, in some embodiments. Inclination angles of a first and second sharp edges of a sharp region may be greater than inclination angles of a first and second blunt edge of a blunt region, respectively, with respect to a longitudinal direction of a cannula at a first and second ridges, according to some embodiments. In some embodiments, at least part of a blunt region distal to a tip end of an outer circumference may be blast-treated for reducing coring. And in some embodiments, a sharp region extending from a tip end of an outer circumference may not be blast-treated.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

Some embodiments of the disclosure may be understood by referring, in part, to the present disclosure and the accompanying drawings, wherein:

FIG. 1 is a plan view of a puncture needle according to a specific example embodiment of the disclosure;

FIG. 2A is a side view of the puncture needle shown in FIG. 1;

FIG. 2B is a side view of the puncture needle shown in FIG. 1;

FIG. 3 is a cross-sectional view taken along line in FIG. 2A;

FIG. 4 is a cross-sectional view taken along line IV-IV in FIG. 2A;

FIG. 5 is a cross-sectional view taken along line V-V in FIG. 2A;

FIG. 6 is a photograph of a plane of a puncture needle following a blast treatment according to a specific example embodiment of the disclosure;

FIG. 7 is an enlarged planar photograph of a jaw portion of the puncture needle shown in FIG. 6;

FIG. 8 is an enlarged perspective photograph of the jaw portion of the puncture needle shown in FIG. 6;

FIG. 9 is an enlarged planar photograph of a jaw portion of a puncture needle that is not subjected to blast treatment;

FIG. 10 is an enlarged perspective photograph of the jaw portion of the puncture needle shown in FIG. 9;

FIG. 11 is an enlarged planar photograph of the jaw portion of the puncture needle shown in FIG. 9;

FIG. 12 is an enlarged perspective photograph of the jaw portion of the puncture needle that is not subjected to blast treatment;

FIG. 13 is an enlarged planar photograph of a side removal portion of the puncture needle according to a specific example embodiment of the disclosure;

FIG. 14 is an enlarged perspective photograph of the side removal portion of the puncture needle shown in FIG. 13;

FIG. 15 is an enlarged planar photograph of a side removal portion of a puncture needle that is not subjected to blast treatment;

FIG. 16 is an enlarged perspective photograph of the side removal portion of the puncture needle shown in FIG. 15;

FIG. 17 is an enlarged planar photograph of the side removal portion of the puncture needle shown in FIG. 15;

FIG. 18 is an enlarged perspective photograph of the side removal portion of the puncture needle shown in FIG. 15;

FIG. 19 is a view showing a nozzle shift according to a specific example embodiment of the disclosure;

FIG. 20 is a view showing a nozzle shift according to a specific example embodiment of the disclosure;

FIG. 21 is a photograph showing blast masking according to a specific example embodiment of the disclosure;

FIG. 22 is a photograph showing a blast treatment range before nozzle shift (heavy line) according to a specific example embodiment of the disclosure;

FIG. 23 is a photograph showing a blast treatment range before (heavy line) and after (thin line) nozzle shift according to a specific example embodiment of the disclosure;

FIG. 24 is a plan view of a puncture needle showing a blast masking range (M2=30%) according to a specific example embodiment of the disclosure;

FIG. 25 is a side view of a puncture needle in which a ratio between a length M1 of a region subjected to blast treatment and a length M2 of a region not subjected to blast treatment is 50:50 according to a specific example embodiment of the disclosure;

FIG. 26 is a plan view of a puncture needle in which the ratio between length M1 of the region subjected to blast treatment and length M2 of the region not subjected to blast treatment is 60:40 according to a specific example embodiment of the disclosure;

FIG. 27 is a side view of a puncture needle in which a ratio between a length Si of first surface 10 and a length S2 of second surface 20 is 65:35 according to a specific example embodiment of the disclosure;

FIG. 28 is a front view showing cannula 6 as a whole and hub 60 for holding cannula 6 according to a specific example embodiment of the disclosure;

FIG. 29 is a cross-sectional view of an embedded port according to a specific example embodiment of the disclosure;

FIG. 30 is a graph showing the force necessary for insertion of the puncture needle according to a specific example embodiment of the disclosure;

FIG. 31 is a side view of a puncture needle according to a specific example embodiment of the disclosure; and

FIG. 32 is a plan view of the puncture needle shown in FIG. 31.

Table 1 below includes the reference numerals used in this application. The thousands and hundreds digits correspond to the figure in which the item appears while the tens and ones digits correspond to the particular item indicated. Similar structures share matching tens and ones digits.

TABLE 1

| Reference | |
|---|---|
| Numeral | Description |
| 1 | puncture needle |
| 1a | longitudinal direction |
| 2 | blade surface |
| 3 | lumen |
| 4 | puncture portion |
| 6 | cannula |
| 10 | first surface |
| 20 | second surface |
| 21 | right-side second surface |
| 22 | left-side second surface |
| 23 | center line |
| 29 | thick portion |
| 31 | tip portion |
| 32 | base end portion |
| 33 | ridge |
| 34 | ridge |
| 35 | inner circumferential edge |
| 36 | outer circumferential edge |
| 37 | edge |
| 39a | blunt region |
| 39b | sharp region |
| 50 | nozzle |
| 60 | hub |
| 61 | elongate body |
| 62 | angular tip |
| 63 | needle wall |
| 64 | blade |
| 65 | first zone |
| 66 | second zone |
| 66a | first phase of a second zone |
| 66b | second phase of a second zone |
| 67 | third zone |
| 68 | blade surface |
| 69 | outer |
| 70 | inner |
| 71 | central longitudinal axis |
| 72 | first phase portion of a blade surface |
| 73 | second phase portion of a blade surface |
| 201 | septum |
| 202 | housing |
| 203 | reservoir |
| 204 | outlet tube |
| 205 | catheter |
| X | first plane |
| Y | second plane |
| Z | third plane |

### DETAILED DESCRIPTION

The present disclosure relates, in some embodiments, to systems, apparatuses, and/or methods for accessing a space (e.g., a lumen) using an annular tool (e.g., a needle) with reduced (e.g., without or substantially without) barrier material being released in the cavity defined by the annulus of the tool (e.g., within the cavity defined by the wall of the needle). According to some embodiments, an annular tool (e.g., a needle) as described herein may be used in connection with any medical, therapeutic, or other treatment of subjects (e.g., human or animals) For example, an annular tool (e.g., a needle) may be used as or used to create or provide a subcutaneous injection port, an infusion line, a mixed injection tube in a blood circuit, a blood collection tube, a chemical container such as a vial container, and/or combinations thereof.

### 1. Structure of Puncture Needle

According to some embodiments, a puncture needle may comprise a cannula provided with a lumen. A cannula may comprise a blade surface, the blade surface including: a sharp region located on a tip side of the blade surface and provided with a sharp edge on inner and outer circumferences of the blade surface; and a blunt region located on a base end side of the blade surface and provided with a blunt edge on the inner and outer circumferences of the blade surface, and a tip of the blade surface is provided with a thick portion formed by a boundary between two regions of the blade surface, and the boundary forms an edge.

A vertex of a thick portion may form, in some embodiments, the edge, while such edge is not formed in a one-plane-cut blade surface. Due to the shape of the tip, a rubber stopper may be cleaved along the blade surface. Thereafter, when the rubber stopper reaches the blunt edge portion, the rubber becomes less likely to break and is cleaved. As a result, coring may be suppressed. Furthermore, since a thick portion is provided, a cannula may have increased strength according to some embodiments. The rubber is less likely to break on the outside since, in some embodiments, the outside of the cannula is blunt.

In some embodiments, a blunt region is configured to extend over a half or more of a height of a cannula. Accordingly, a cleaving end formed in the rubber stopper is less likely to become larger than a semicircle and thereby a rubber stopper may be less likely to break.

According to some embodiments, a blade surface a blade surface may have a first surface located on a base end side and a second surface located on a tip side, and a side surface ridge line which is a boundary between a first surface and a second surface may be located in a blunt region. When a rubber surface hits a sharp side surface ridge line, the rubber may deflect and may be cleaved excessively. However, when a side surface ridge line is blunt, cleaving may be less likely to occur.

In some embodiments, a length of a sharp region is less than or equal to 40% of a length of a blade surface. Since a cleaving end formed in the rubber stopper may be reduced in size, a rubber stopper may be less likely to break and the needle can pass through the rubber stopper.

According to some embodiment, a blunt region may be formed by blast treatment, and an area of a surface subjected to blast treatment is larger on a base end side than on a tip side on a first surface, and larger on the base end side than on the tip side on the second surface (i.e., the blast treatment is performed more intensely on the base end side than on the tip side). Therefore, the blade surface of the second surface may have a shape having a curvature that becomes gradually smaller from the tip side toward the base end side. In a blunt region, a blade surface may be formed to become gradually blunter toward the base end direction, and thus, coring may be suppressed more reliably.

Numerical ranges in each configuration are provided below. Some of these ranges may be preferred for certain applications in light of the coring results obtained in tests, the results of which are shown in Tables 2 and 3.

In some embodiments, a second surface may have a right-side second surface and a left-side second surface, and an angle formed by the right-side second surface with respect to the left-side second surface may be greater than or equal to 66° and less than or equal to 114°.

According to some embodiments, a ratio of a length of a second surface to a length of a blade surface may be greater than or equal to 35% and less than or equal to 50%.

A blast treatment, in some embodiments, may not be performed in a range where a length from a tip of a blade surface is greater than or equal to 30% and less than or equal to 40% of a length of the blade surface.

In some embodiments, an angle formed by a right-side second surface with respect to a left-side second surface may be greater than or equal to 66° and less than or equal to 94°.

According to some embodiments, a length of a second surface may be greater than or equal to 46% and less than or equal to 54% of a length of a blade surface.

An angle formed by a right-side second surface with respect to a left-side second surface may be greater than or equal to 66° and less than or equal to 74°, according to some embodiments.

In some embodiments, a blast treatment may not be performed in a range where a length from a tip of a blade surface is greater than or equal to 26% and less than or equal to 34% of a length of the blade surface.

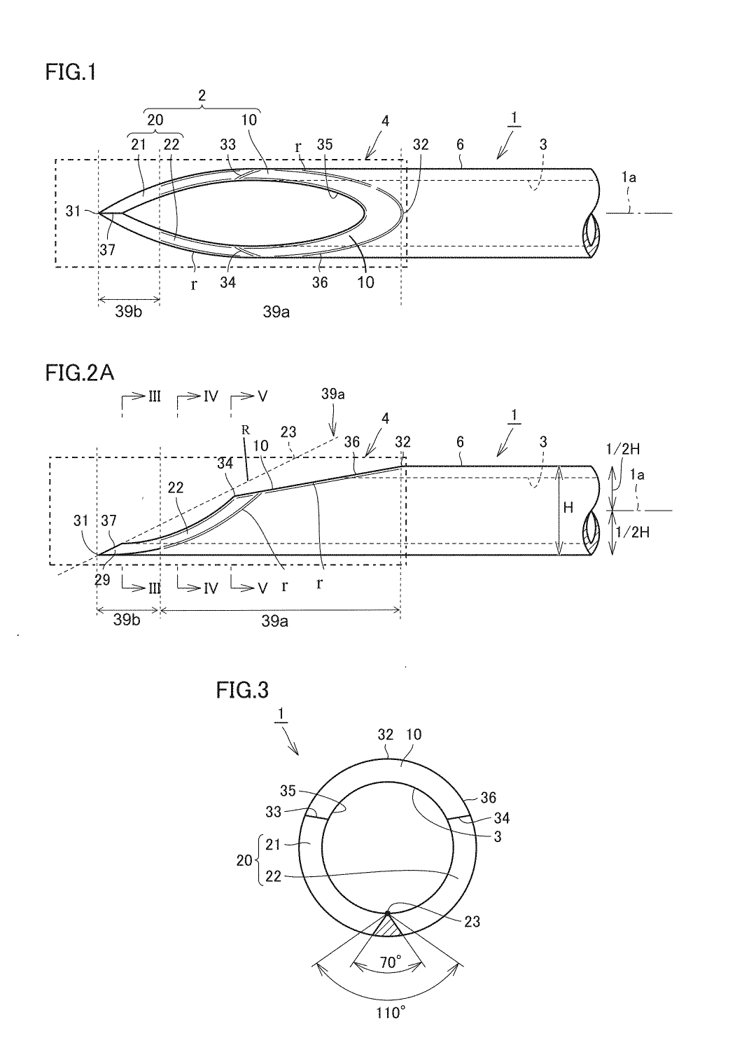

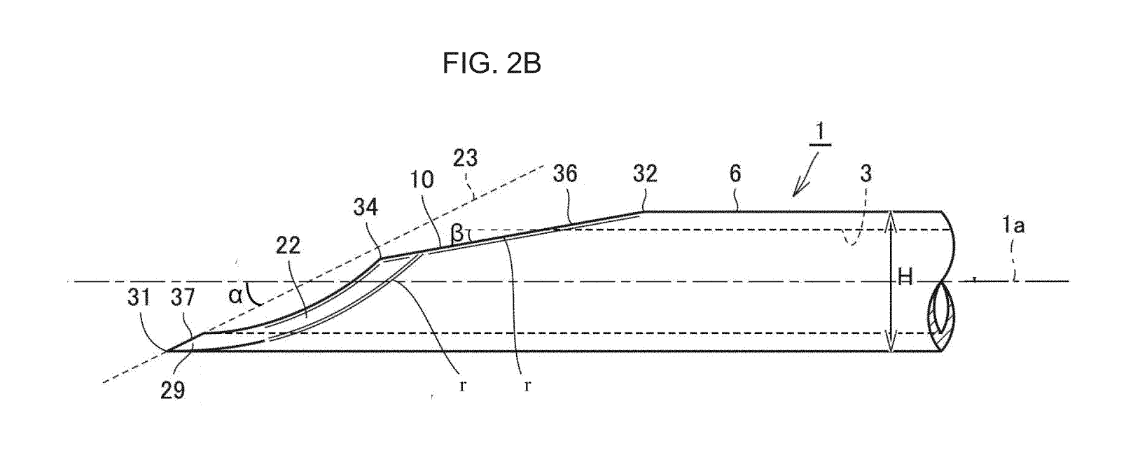

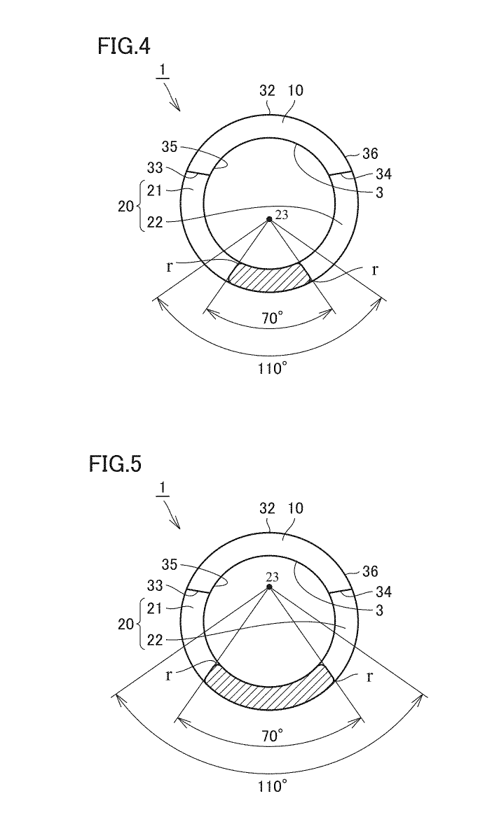

FIG. 1 illustrates a plan view of a puncture needle according to a specific example embodiment of the disclosure. FIG. 2A illustrates a side view of the puncture needle shown in FIG. 1. FIG. 2B illustrates a side view of the puncture needle shown in FIG. 1. FIG. 3 illustrates a cross-sectional view taken along line III in FIG. 2A. FIG. 4 illustrates a cross-sectional view taken along line IV in FIG. 2A. FIG. 5 illustrates a cross-sectional view taken along line V in FIG. 2A.

As shown in FIGS. 1 to 5, a puncture needle 1, according to some embodiments, may be provided with a puncture portion 4 having a blade surface 2 inclined with respect to longitudinal direction 1a, as shown by an alternating long and short dashed line. In some embodiments, puncture needle 1 may comprise cannula 6, and cannula 6 may be further provided with lumen 3. An opening surface of lumen 3 may be defined by an inner circumferential edge 35, according to some embodiments. An outer circumferential edge 36 of lumen 3 may be formed on an outside of an inner circumferential edge 35. For example, inner circumferential edge 35 and outer circumferential edge 36 of inclined blade surface 2 may be formed into an “r” (lower case) shape as a blunt region. Both inner circumferential edge 35 and outer circumferential edge 36 have a substantially oval shape, according to some embodiments. In some embodiments, an “r” (lowercase) shape may be formed by blast treatment. The “r” (lower case) shape is not formed on the side close to a tip portion 31. The “r” (lower case) shape is formed on the side close to a base end portion 32.

As shown in FIG. 1, in some embodiments, inclined blade surface 2 may have a first surface 10 on a base end side and second surface 20 on a tip side connecting to first surface 10. As shown in FIG. 2B, first surface 10 may be inclined at a first angle, β, with respect to lumen 3. Lumen 3 may be parallel to longitudinal direction 1a (shown by alternate long and short dash line) of cannula 6, in some embodiments. In some embodiments, first surface 10 may have a right-side first surface and a left-side first surface.

According to some embodiments, second surface 20 may be configured to connect to first surface 10. Second surface 20 may include right-side second surface 21 and left-side second surface 22. Second surface 20 define a convex curve relative to center line 23. Ridge 33 may form a boundary between right-side first surface 10 and right-side second surface 21, in some embodiments. In some embodiments, ridge 34 may form a boundary between left-side first surface 10 and left-side second surface 22. Right-side second surface 21 and left-side second surface 22 may each comprise a center line 23. An edge 37 may be formed at a boundary between right-side second surface 21 and left-side second surface 22. As shown in FIG. 2B, center line 23 may be formed by edge 37 and may form an angle, α, with respect to longitudinal direction 1a (shown by alternate long and short dash line) of cannula 6, in some embodiments. In some embodiments, a tip of blade surface 2 may comprise a thick portion 29 formed by a boundary between a right-side second surface 21 and a left-side second surface 22 with the boundary forming edge 37. A section of a tip of blade surface 2 taken perpendicular to longitudinal direction 1a may have the shape of a partial Reuleaux triangle in which the base is a circular segment of radius (“Rx”) (e.g., radius equals the thickness of the cannula wall) and the two sides extending (e.g., two sides of equal length) from each end of the base are straight, as shown in FIGS. 4 and 5. In some embodiments, second surface 20 (even if curved) may be generally inclined at a second angle with respect to longitudinal direction 1a of cannula 6. As shown in FIGS. 4 and 5, illustrating cross-sectional views of FIG. 2A cut along lines IV and V respectively, an angle formed by right-side second surface 21 and left-side second surface 22 is preferably greater than or equal to 70° and less than or equal to 110°, according to some embodiments. A ratio between a length of first surface 10 and a length of second surface 20 may be 50:50, in some embodiments.

In some embodiments, ridge 33, and ridge 34, and at least a portion of a first surface 10 may be subjected to blast treatment. All parts of a first surface 10 may be subjected to blast treatment in some embodiments. According to some embodiments, a blast treatment may be performed more intensely on a base end side than on a tip side of first surface 10, and a degree of cutting by the blast treatment may be higher on the base end side than on the tip side. A region near tip portion 31 is not subjected to blast treatment, in some embodiments. An edge 37 is formed at a boundary between right-side second surface 21 and left-side second surface 22. According to some embodiments, edge 37 is not subjected to a blast treatment. In some embodiments, a surface area subjected to blast treatment on a base end side is larger than an area subjected to blast treatment on a tip end side.

According to some embodiments, a new edge may be formed by a right-side first surface intersecting a left-side first surface at an angle. A new edge may be subjected to blast treatment, according to some embodiments.

As shown in FIG. 1 and FIG. 2A, blade surface 2 may comprise a sharp region 39b disposed on a tip side of blade surface 2. In some embodiments, sharp region 39b may comprise a sharp edge disposed on an inner circumference of a tip side of blade surface 2 and a sharp edge disposed on an outer circumference of the tip side of blade surface 2. Blade surface 2, in some embodiments, may comprise blunt region 39a disposed on a base end side of blade surface 2. According to some embodiments, blunt region 39a may comprise a blunt edge disposed on an inner circumference of a base end side of blade surface 2 and a blunt edge disposed on an outer circumference of the base end side of blade surface 2.

As shown in FIGS. 2A and 2B, where H represents a height of cannula 6 (direction orthogonal to a central axis shown by alternate long and short dash line 1a), blunt region 39a may be greater than or equal to 1/2 H. In some embodiments, first surface 10, second surface 22, and center line 23 together define an “R” (upper case) shape as illustrated in FIG. 2A.

As shown in FIGS. 4 and 5, in some embodiments, inner circumferential edge 35 and outer circumferential edge 36 are configured to form a cross-section having an “r” shape. Forming a cross section having an “r” (lower case) shape may reduce cleaving of a septum and thereby reduce coring or a formation of a core piece.

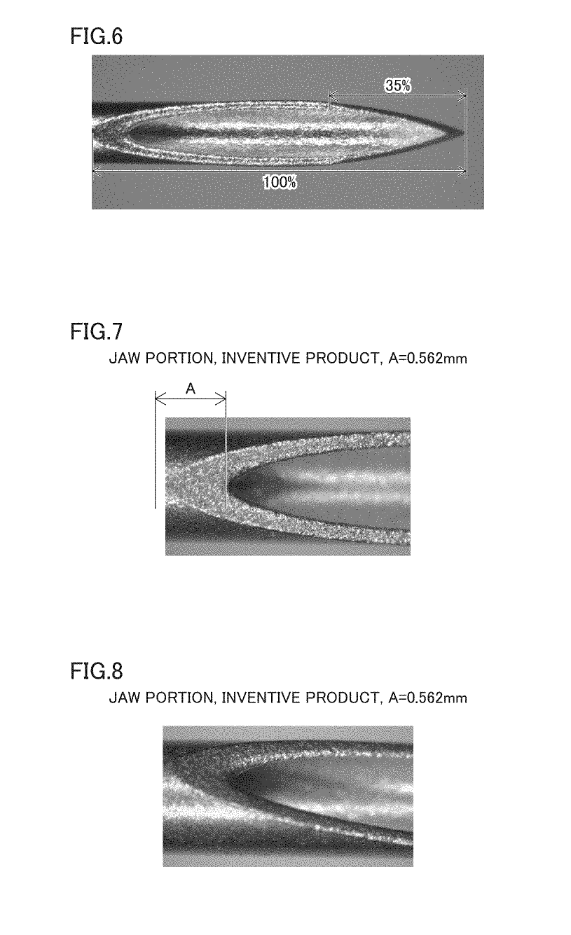

In some embodiments, as shown in FIG. 6, a length of second surface 20 may 35% of a length of blade surface 2. According to some embodiments, an “r” (lower case) shape may be formed by a blast treatment. Referring to FIGS. 7 and 8, a length A of a jaw portion subjected to blast treatment is 0.562 mm in some specific example embodiments.

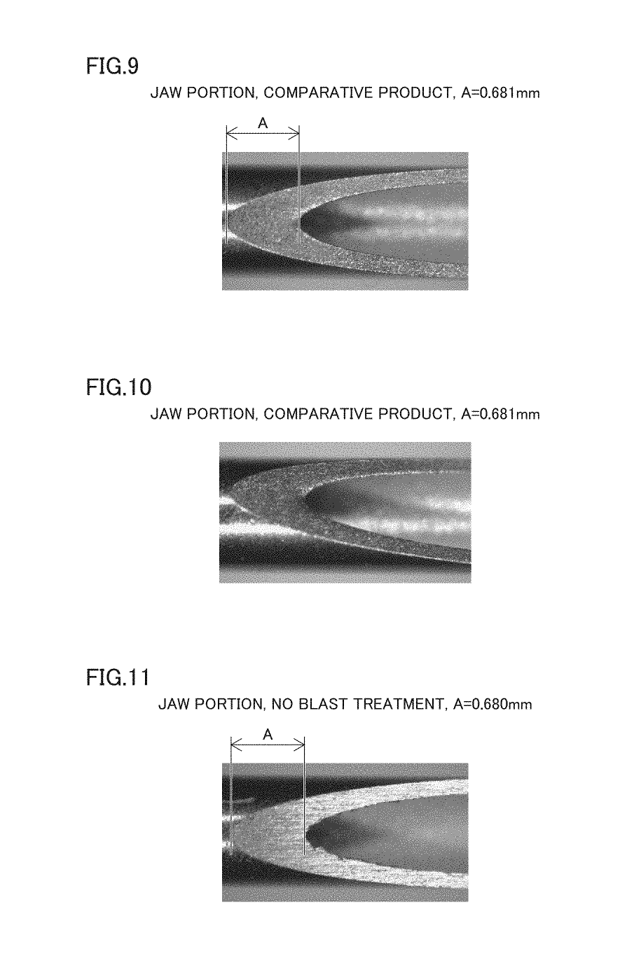

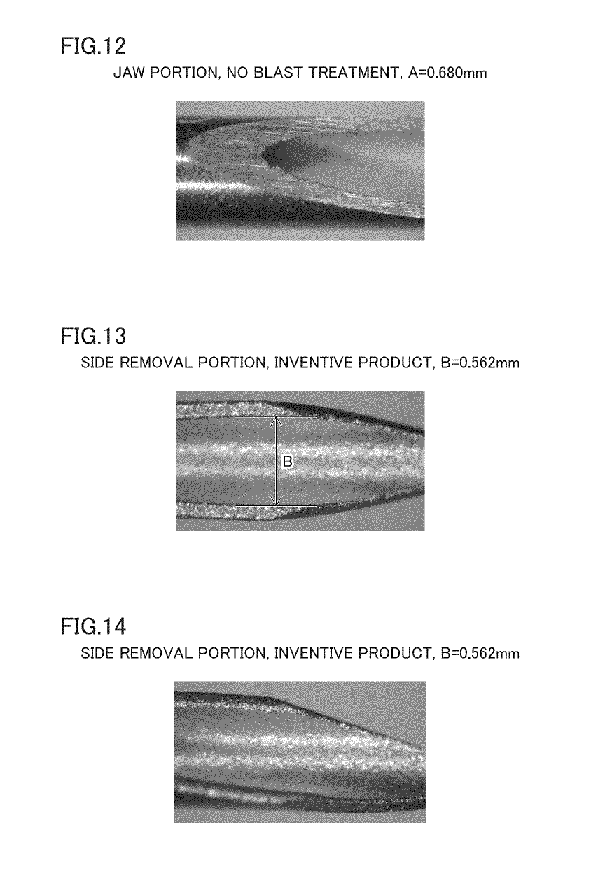

As shown in FIG. 6, a blade surface of a second surface may have a curvature that becomes smaller from the tip toward the base end. In other words, the blade surface of the second surface is the blade surface in which an area of the blunt region becomes gradually larger from the tip toward the base end. FIGS. 9 and 10 illustrate a comparative product without a blast treatment having a length A of a jaw portion of 0.681 mm FIGS. 11 and 12 illustrate embodiments of the present disclosure prior to a blast treatment and having a length A of a jaw portion of 0.680 mm.

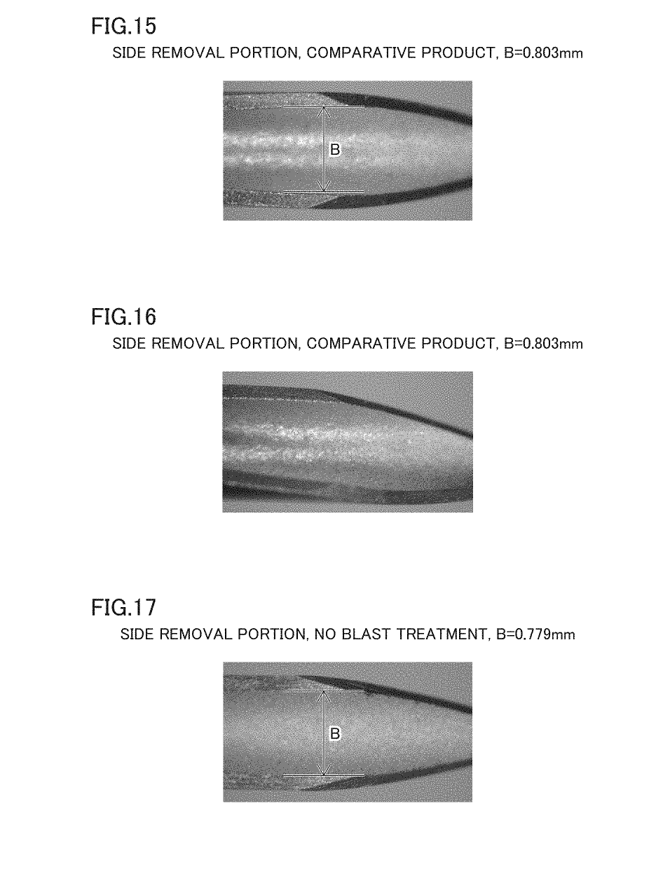

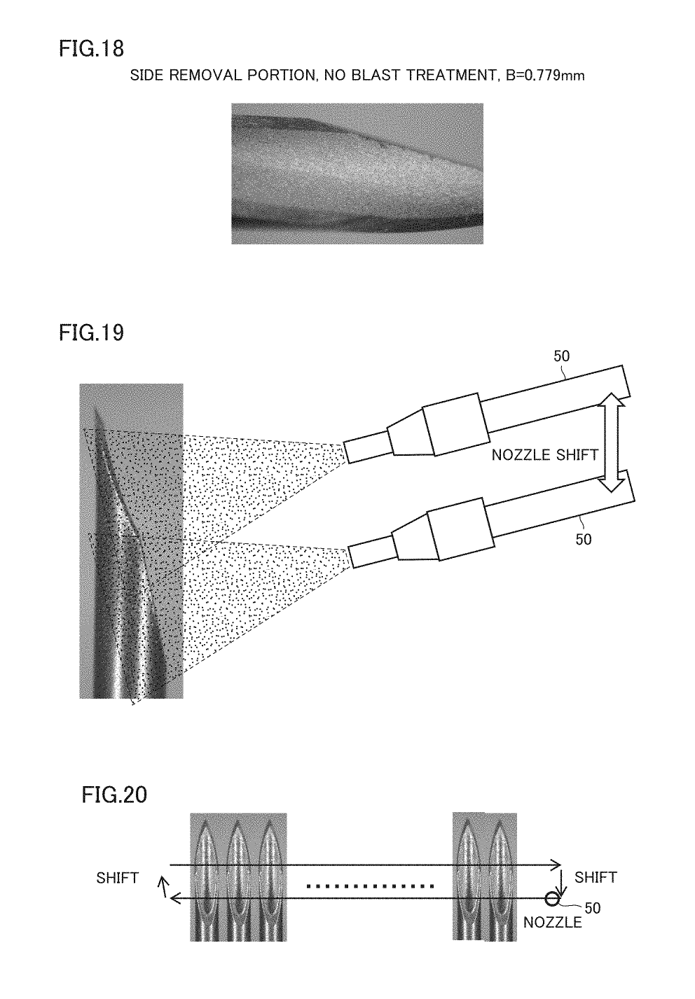

FIGS. 15 and 16 illustrate a comparative product without a blast treatment having a side removal portion with a width B of 0.803 mm FIGS. 17 and 18 illustrate embodiments of the present disclosure prior to a blast treatment having a side removal portion with a width B of 0.779 mm FIGS. 13 and 14 illustrate embodiments of the present disclosure subsequent to a blast treatment, in which width B of a side removal portion of 0.562 mm.

FIG. 28 shows a cannula 6 and a hub 60 for holding cannula 6. Cannula 6 exposed from hub 60 has a linear shape as a whole. Cannula 6 may have an L shape, in some embodiments. In some embodiments, a cannula 6 may have a shape that is configured to be bent in a non-linear shape, but is not configured to bend more than 90 degrees (e.g., L-shaped).

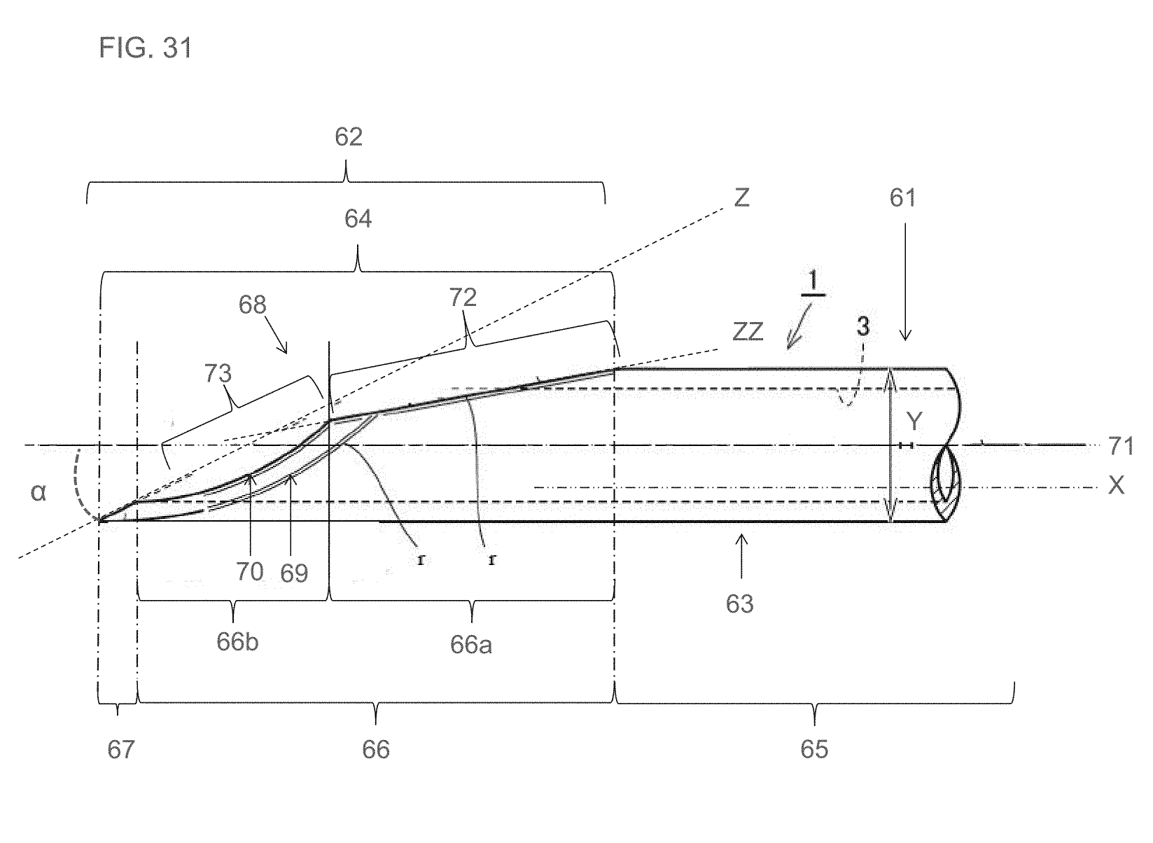

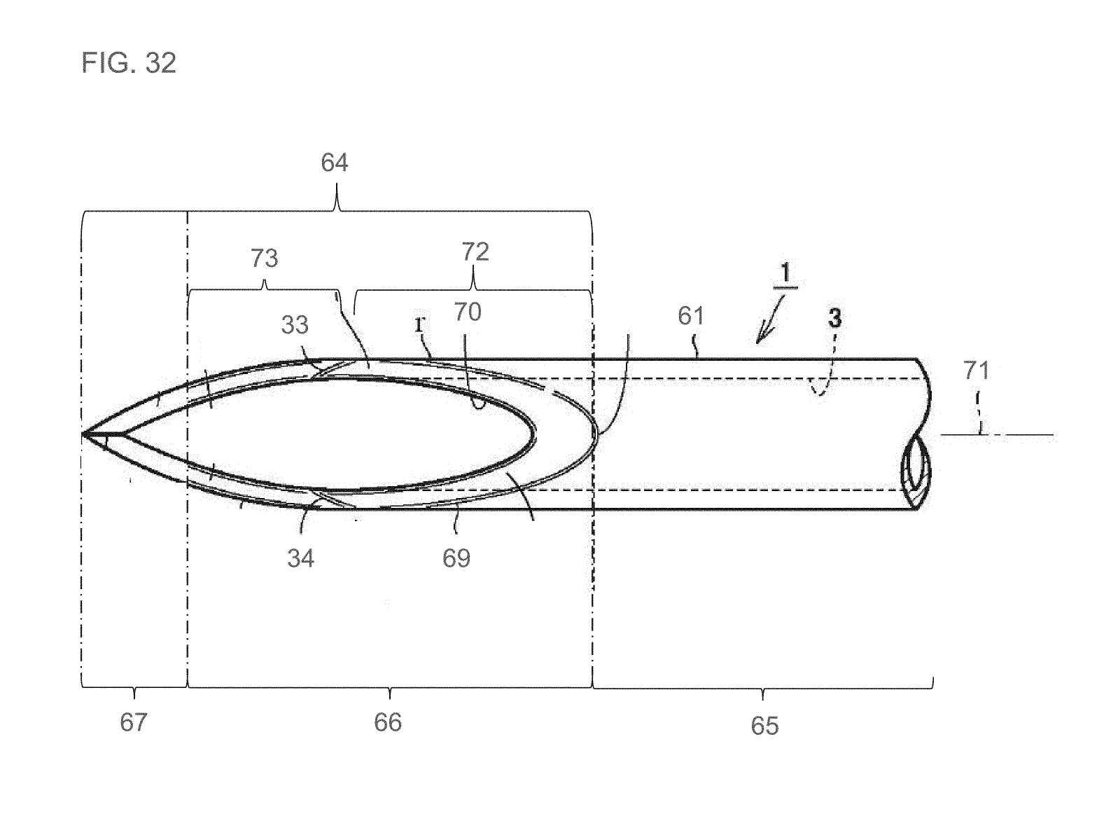

As illustrated in FIGS. 31 and 32, a puncture needle 1 may include, according to some embodiments, an elongate body 61 and/or an angular tip 62 at one or both ends. In some embodiments, a puncture needle 1 may comprise a needle wall 63, at least a portion of which may have an annular configuration encircling and/or defining a lumen 3 with a diameter, a length, and a central longitudinal axis. A needle wall 63 and the lumen it defines, lumen 3 may have an opening at one or both ends. A puncture needle 1 may comprise, in some embodiments, a blade 64 configured to facilitate insertion into and/or through a barrier material.

According to some embodiments, a puncture needle may have a needle wall defining a central longitudinal axis 71 positioned within a first plane X and also within a second plane Y that perpendicularly intersects the first plane. Thus, the axis is positioned at the intersection of these two planes. A puncture needle may include three zones along its length. For example, as shown in FIG. 31, a first zone 65 may have a circumferentially contiguous tubular configuration and fully encircle a central longitudinal axis. A first zone may be configured to have a portion of the needle wall defining a lumen 3 and having an inner tubular wall with an inner tubular circumference and an inner tubular wall with an outer tubular surface. The radius of the inner and outer wall may be constant along the length of a first zone. For example, the inner wall may be free of wall spurs, bumps, ridges, or other projections into the lumen.

A second zone 66 may have a wall that only partially encircles (e.g., with respect to its outer circumference) a central longitudinal axis. A second zone 66 may have two distinct phases. A first phase of a second zone 66a may adjoin and extend contiguously from a first zone 65. A first phase of a second zone 66a may be configured to have a portion of the needle wall 63 with a lateral opening defining a first phase portion of a blade surface 72. A first phase portion of a blade surface 72 may have an outer blade circumference 69 (e.g., contiguous with the outer needle wall surface) and an inner blade circumference 70 (e.g., contiguous with the inner needle wall surface). A first phase portion of a blade surface may be within a third plane Z oblique to a central longitudinal axis and a first plane X and generally perpendicular to a second plane Y.

In some embodiments, radii extending to the inner surface and outer surfaces of needle wall along the length of a first phase of a second zone 66a may be (1) equal to radii extending to the inner and outer surface of needle wall along the length of the first zone, respectively, and/or (2) constant along the length of the first phase of a second zone. For example, the inner wall may be free of wall spurs, bumps, ridges, or other projections into the lumen.

A second phase of a second zone 66b may adjoin and extend contiguously from a first phase of a second zone 66a. A second phase of a second zone 66b may be configured to have a portion of the needle wall with a lateral opening defining a second phase portion of a blade surface 73. A second phase portion of a blade surface 73 may be configured to be contoured rather than planar. For example, a second phase portion of a blade surface 73 may be configured to generally convex relative to a central longitudinal axis and/or a line extended from and along tip edge (described below). A second phase portion of a blade surface 73 may define a contoured strip, the curvature of which changes along the length of second phase of a second zone 66b. Any tangent to a contoured strip may be oblique to a central longitudinal axis 71, oblique to a first plane X, oblique to a second plane Y, and oblique to a fourth plane ZZ.

In some embodiments, radii extending to the inner surface and outer surfaces of needle wall along the length of a second phase of a second zone may be respectively tapered relative to radii extending to the inner surface and outer surfaces of needle wall along the length of a first phase of a second zone. For example, as may be seen in FIG. 32, inner radius r₁>inner radius r₂>inner radius rₙ, such that the inner surface is tapered. The inner wall may be free of wall spurs, bumps, ridges, or other projections into the lumen.

A first phase portion of a blade surface 72 may be contiguous with a second phase portion of a blade surface 73. Similarly an inner circumference and outer circumference of a first phase portion of a blade surface may be contiguous with, respectively, an inner circumference and an outer circumference of a second phase portion of a blade surface. Together, an inner circumference of a first phase portion of a blade surface and an inner circumference of a second phase portion of a blade surface may define an inner edge of a blade surface. Together, an outer circumference of a first phase portion of a blade surface and an outer circumference of a second phase portion of a blade surface may define an outer edge of a blade surface. An inner edge and an outer edge each may independently have any desired curvilinear shape. For example, an inner edge and an outer edge may form generally concentric curvilinear shapes (e.g., leaf, teardrop, and/or folium), each having a tip positioned at its most distal extent. An inner edge may define an opening to a needle lumen. In some embodiments, a blade may comprise at least a portion of an inner edge and/or at least a portion of an outer edge.

An inner circumference and/or an outer circumference within a first phase portion of a blade surface 72 may have, as may be seen in FIG. 32, two ends and a parabolic shape, partial oval shape, an “∩” shape, semi-circular shape or any other desired shape. An inner circumference and/or an outer circumference within a second phase portion of a blade surface 73 may have two ends and a “V” shape, a rounded “V” shape, as may be seen in FIG. 32, or any other desired shape. The ends of a first phase portion of a blade surface and a second phase portion of a blade surface may be joined to form opposing lateral ridges 33/34. Each lateral ridge may independently define a line oblique to the first and second planes and, optionally also oblique to the third plane. Each lateral edge may extend between an inner surface and an outer surface. Lateral ridges may be positioned on opposite or generally opposite sides of a needle wall opening. In some embodiments, opposing ridges may be toed in, wherein a proximal end (e.g., positioned closer to a first zone of the puncture needle) of a ridge is adjacent to an outer surface of a needle wall and a distal end (e.g., positioned farther from a first zone of the puncture needle) of a ridge is adjacent to an inner surface of the needle wall. Opposing ridges may be coplanar, according to some embodiments. Opposing ridges may be positioned (1) more distally along a central axis so a first phase of a second zone is larger than a second phase of the second zone, (2) more proximally along a central axis so a first phase of a second zone is smaller than a second phase of the second zone, or (3) so a first phase of a second zone is equal or substantially equal to a second phase of the second zone.

In some embodiments, sections of a second phase of a second zone taken perpendicular to a central longitudinal axis (e.g., anywhere along the length of the second phase of the second zone) may have an annular sector shape defined by an outer wall surface sector, an inner wall surface sector, and a left and right blade surface. Lines extending from a left and right blade surfaces intersect to form an angle θ, which, in some embodiments may be from about 70° to about 110°. According to some embodiments, an angle θ may be constant through successive sections along up to the full length of the second phase of the second zone. An angle θ may vary, in some embodiments, through successive sections along up to the full length of the second phase of the second zone.

A third zone 67 may be configured to be a needle tip. A third zone 67 may have a wall that only partially encircles (e.g., with respect to its outer circumference) a central longitudinal axis. A third zone may adjoin and extend contiguously from a second phase of a second zone. A section of a tip perpendicular to a longitudinal axis taken anywhere in the third zone (i.e., between a puncture needle's distal extent and the beginning of the third annular sector zone) may define a partial Reuleaux triangle in which one side is a circular segment of radius (“Rx”) (e.g., radius equals the radius of the inner surface of the cannula wall) and two sides (e.g., two sides of equal length) are straight. The two straight sides define an angle α^{tip}. The radius (r) and angle α^{tip} are constant in successive sections taken along the length of the second zone. The length of the two straight sides and the circumference of the circular segment generally decrease (but r and α^{tip} remain constant) in successive sections perpendicular to the longitudinal axis moving toward the distal extent of the tip. Each vertex formed by the straight sides (blade surfaces) in successive sections defines a tip edge. A tip edge and a central longitudinal axis may form an angle α. A third zone may begin where sections taken perpendicular to a central longitudinal axis transition from having an annular sector shape to a partial Reuleaux triangle (or vice versa).

### 2. Method of Manufacturing a Puncture Needle

The present disclosure relates, in some embodiments, to methods for making a puncture needle. For example, hollow puncture needle 1 for medical purposes, according to some embodiments of the disclosure, may be manufactured in accordance with the following method.

### 2-1 Selection of Material

According to some embodiments, a cannula (e.g., cannula 6) may be selected (e.g., as a raw tube subjected to primary molding processing). In some embodiments, a cannula may comprise any desired material including, for example, iron, steel, stainless steel, composites, and combinations thereof. A length, an inner diameter, and an outer diameter of a cannula may be selected in accordance with a target end product.

### 2-2 Grinding

According to some embodiments, a cannula may be processed to form a blade surface (e.g., blade surface 2 having first surface 10, right-side second surface 21, and left-side second surface 22). In some embodiments, processing may include grinding. For example, a disc-shaped (cylindrically-shaped) rotary grindstone having a prescribed thickness may be placed on a tip side of a cannula (e.g., cannula 6) and above a target first surface 10. Then, a rotation central axis of the rotary grindstone may be set in parallel with the target surface (e.g., first surface 10). The rotary grindstone may be arranged to produce a surface (e.g., first surface 10) having an angle (first angle) formed by the surface and a longitudinal axis (e.g., longitudinal direction 1a) of, for example, 10°. The rotary grindstone thus arranged may be rotationally driven around the rotation central axis thereof. In some embodiments, an entire tip surface of a cannula (e.g., cannula 6) may be subjected to grinding by an outer circumferential surface of a rotary grindstone, forming a first surface 10.

According to some embodiments, a cannula may be subjected to further processing by changing a relative position of a rotary grindstone relevant to the cannula (e.g., cannula 6) and grinding an additional tip surface. In some embodiments, a position of a rotary grindstone may be fixed, while a position of a cannula being worked may be non-fixed (e.g., alterable).

In some embodiments, a cannula (e.g., cannula 6) and a rotary grindstone may be positioned relative to each other to form a second surface. For example, a cannula may be inclined such that an angle formed by a second surface with respect to longitudinal axis la is, for example, 18°. According to some embodiments, a cannula (e.g., cannula 6) may be circumferentially rotated counterclockwise by, for example, 55° around a line extending from and along a tip edge (e.g., center line 23) with respect to the rotary grindstone from a position where first surface 10 has been formed. A rotary grindstone may be brought into contact with the cannula to form a right-side second surface 21 by processing (e.g., grinding).

A cannula (e.g., cannula 6) may be circumferentially rotated clockwise by, for example, 110° around a line extending from and along a tip edge (e.g., center line 23) with respect to the rotary grindstone from a right-side second surface (e.g., right-side second surface 21). In some embodiments, a rotary grindstone may be brought into contact with a cannula (e.g., cannula 6) to form a left-side second surface (e.g., right-side second surface 22) by processing (e.g., grinding). As a result, a puncture needle may have having a first angle (e.g., 10°), a second angle (e.g., 18°), and a rotation angle (e.g., 110°).

### 2-3 Blast Treatment

According to some embodiments, at least a portion of (but less than all of) a blade surface may be subjected to a treatment to roughen the surface and/or grind down sharp edges. For example, at the time when first surface 10 and second surface 20 are formed by the grinding processing, inner circumferential edge 35, outer circumferential edge 36 and ridges 33 and 34 may have a sharp shape, and thus, these are subjected to blast treatment to form an “r” (lower case) shape (curved shape).

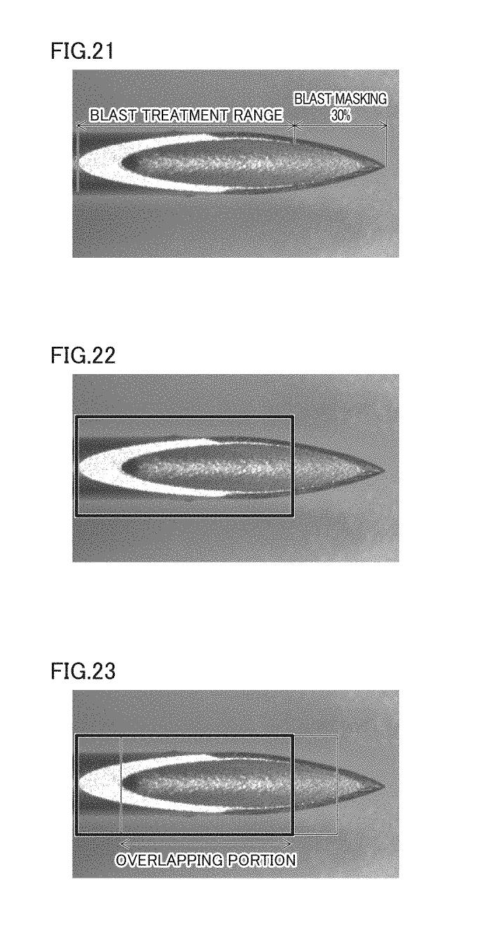

In some embodiments, a blast treatment may comprise propelling an abrasive material through a nozzle (e.g., nozzle 50) toward a surface to be treated. A blast treatment may include shifting a nozzle (e.g., nozzle 50), for example as shown in FIGS. 19 and 20, to emit a powder onto a puncture needle. As shown in FIGS. 21-23, in some embodiments a tip portion (e.g., a region where a length is 30% of that of the blade surface) of a puncture needle may be masked and thereby protected from a blast treatment. Even if the powder is emitted onto the masked region, this portion is not protected from blast treatment. As shown in FIG. 22, in some embodiments a nozzle shift is not used and a blast treatment is performed only in a limited range. In some embodiments, a nozzle shift is used and a blast treatment is performed in a wider range, as shown in FIG. 23.

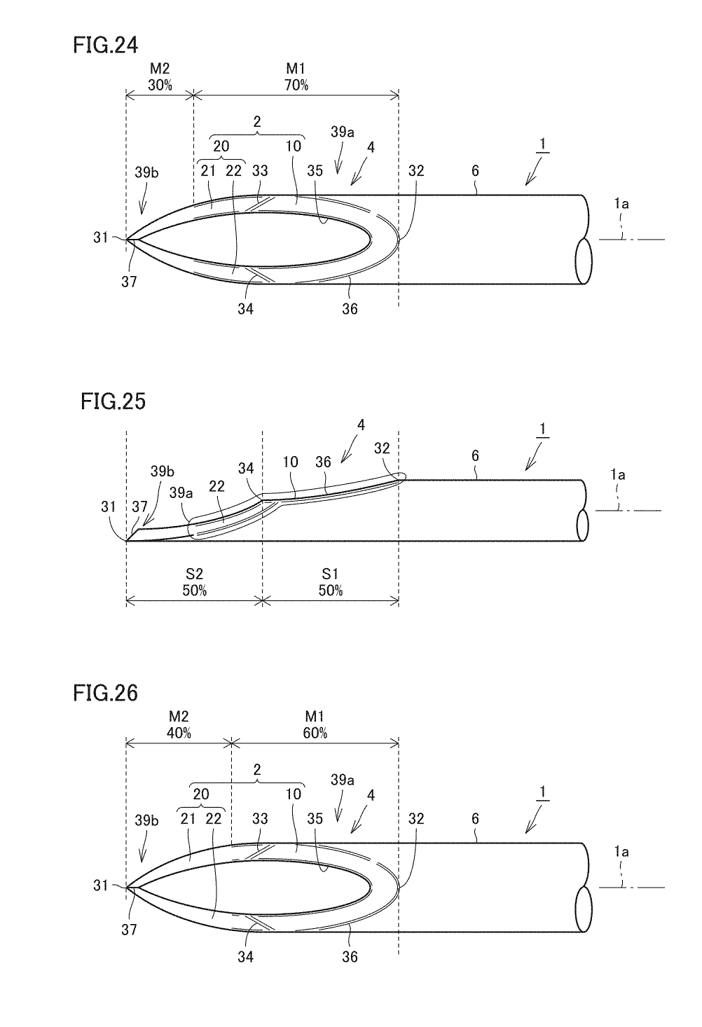

As shown in FIGS. 24 and 25, a blast treatment may be performed more intensely on a side close to base end portion 32 on at least one of first surface 10 and second surface 20. A ratio between a length M1 of a portion subjected to blast treatment and a length M2 of a portion not subjected to blast treatment can be changed as appropriate. A ratio between a length S1 of first surface 10 and a length S2 of a second surface 20 can also be changed as appropriate. A blast region 39a in FIG. 24 indicates the region subjected to blast treatment and a length thereof is 70% of the length of blade surface 2. FIG. 25 shows puncture needle 1 in which a ratio between length M1 of a region subjected to blast treatment and length M2 of a region not subjected to blast treatment is 50:50.

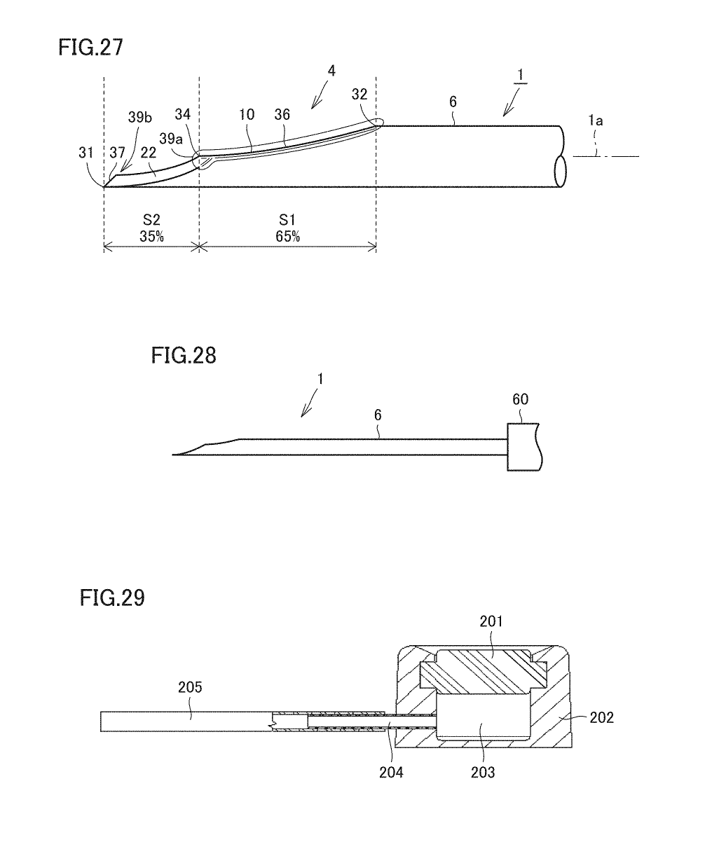

FIG. 26 shows puncture needle 1 in which the ratio between length M1 of a region subjected to blast treatment and length M2 of a region not subjected to blast treatment is 60:40. FIG. 27 shows puncture needle 1 in which a ratio between length 51 of first surface 10 and length S2 of second surface 20 is 65:35.

### 3. Tests

According to some embodiments, any desired test may be used to assess coring performance (e.g., septum material that may be scrapped off or otherwise released) of a puncture needle. One specific example performance test procedure is described below as applied to puncture needle 1.

### 3-1 Coring Test

In medical treatment an embedded port may be used to administer one or more medicaments and/or remove patient fluids. An embedded port refers to a reservoir (e.g., reservoir 203) (normally accompanied with a catheter) placed under the skin as shown in FIG. 29. The embedded port receives the needle through septum 201. The embedded port is often used for administration of a medicament. Reservoir 203 is formed within housing 202. An outlet tube 204 is connected to reservoir 203 and outlet tube 204 is connected to a catheter 205. (e.g., FIG. 29) and is often associated with a housing (e.g., housing 202). Often an outlet tube (e.g., outlet tube 204) is connected to a reservoir (e.g., reservoir 203) and an outlet tube (e.g., outlet tube 204) is connected to a catheter (e.g., catheter 205).

To access a reservoir, a needle may puncture a septum (e.g., septum 201). A septum (e.g., septum 201) may be configured to allow a puncture needle to access a reservoir a plurality of times. In some embodiments, a septum (e.g., septum 201) may comprise an elastic material. During puncture, a core piece may be generated (e.g., a quantity of a septum material generated when a puncture needle makes a hole in a septum). A coring test was performed to evaluate whether use of a needle as described in the present disclosure results in decreased coring (e.g., an absence of a core piece).

Blade surface 2 refers to an inclined portion of the needle. A tip thereof is sharp. Cannula 6 refers to a tube-like portion of the needle through which a liquid passes. A core piece refers to a small piece of the septum material generated when puncture needle 1 makes a hole in a septum 201 (FIG. 29). The jaw portion refers to the cut surface rear side of the needle blade surface, and specifically to a region between inner circumferential edge 35 and outer circumferential edge 36 in base end portion 32. Lumen 3 is defined by an inner surface of cannula 6.

A stylet is a device inserted into lumen 3 to remove the core piece, and is desirably made of metal.

### 3-2 Overview of Test Procedure

An elastic silicon disc (denoted as “septum”) was fixed in a septum holder. Assuming that septum 201 for testing was the embedded port, a tester accessed septum 201 for testing by using the needle. This test was classified into a failure/no-failure test. If the core piece was present in cannula 6, the result was determined as unacceptable.

This test is a test method for determining whether or not the needle is designed and manufactured to prevent the core piece from being generated when the needle accesses septum 201 for testing of the typical port.

The septum was a silicon disc and had a diameter of 0.70±0.01 inches and a thickness of 0.250±0.005 inches. Prior to performing each test, a puncture surface (i.e., a flat surface that a puncture needle would puncture to access a reservoir) was evaluated for pitting and/or chipping. Only a smooth puncture surface was used for core testing. The septum was made of an elastic silicon material having a durometer hardness of 60A (ASTM D2240).

An optical microscope achieving an optical magnification of at least 20 was required. SZ-CTV manufactured by OLYMPUS was used as a main body of the optical microscope and MHF-150L was used as a light source of the optical microscope, and observation was made under a magnification of 20.

### 3-3 Procedure

(1) The needle was taken out of a package. Each needle was tested only one time.

(2) Septum 201 for testing was placed and clamped at a testing machine.

(3) A test tool was fixed to above a cylinder and a member (guide template) for causing the needle to vertically penetrate the septum placed on the test tool.

(4) Puncture needle 1 was inserted into the clamped septum along an outer edge of a circular opening of the guide template. At this time, the blade surface was directed to the circumferential direction as puncture needle 1 was inserted into the septum perpendicularly to the septum surface. The septum was punctured with only one needle at one time. Penetration was performed carefully so as to avoid the previous punctured portion.

(5) It was determined whether or not coring was occurring.

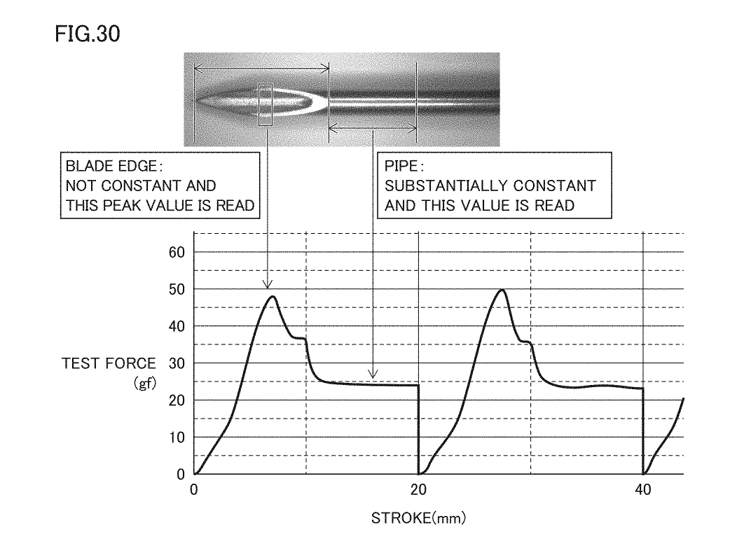

Furthermore, during the coring test, a resistance when puncturing the septum was investigated. The puncture resistance had a resistance of the blade edge and a resistance of the pipe, and these were read as shown in FIG. 30, respectively.

A stylet (a wire) was used to push out of the needle the core piece that may be present in lumen 3. The stylet had an outer diameter that is greater than or equal to 70% (in size) of the inner diameter of cannula 6.

### 4. Results

The following tables show the presence or absence of coring and the puncture resistance. Tables 2 and 3 show the presence or absence of coring. Tables 4 and 5 show the puncture resistance in the first to third lots. Indicated gauge sizes satisfy ISO9626. Specifically, the gauge size of 19 G means that the outer diameter is 1.065±0.035 mm and the inner diameter is 0.704±0.056 mm. The gauge size of 21 G means that the outer diameter is 0.815±0.015 mm and the inner diameter is 0.5185±0.0285 mm. The gauge size of 22 G means that the outer diameter is 0.714±0.016 mm and the inner diameter is 0.415±0.025 mm. All of the puncture needles are made of SUS304.

The first angle refers to the angle formed by first surface 10 and longitudinal direction la (e.g., FIG. 1). The second angle refers to the angle formed by second surface 20 and longitudinal direction 1a (e.g., FIG. 1). The rotation angle refers to the angle formed by right-side second surface 21 and left-side second surface 22 that form second surface 20 (e.g., FIG. 3). A blade surface ratio refers to a ratio of the length of second surface 20 to the entire length of blade surface 2. Blast masking indicates a length of the masked region in second surface 20 with respect to the length of blade surface 2. Blast masking was performed over a prescribed length from tip portion 31 of second surface 20.

TABLE 2

| | | | | | Blade | Blast Condition | | Result | | | |
|---|---|---|---|---|---|---|---|---|---|---|---|
| | | | Second | | Surface | Blast | Nozzle | | | Occurrence | |
| Size | | First Angle | Angle | Rotation Angle | Ratio | Masking | Shift | Cannula Lot | Result | Rate (%) | Core Piece† |
| 19G | 1.05 mm × | 10° | 18° | 110° | 35% | 30% | present | 130724-001 | 0/100 | 0 | — |
| | 42 mm | | | | | | absent | 130724-002 | 9/60 | 15 | large |
| | | | | | | 40% | present | 130724-003 | 8/100 | 8 | large |
| | | | | | | | absent | 130724-004 | 3/30 | 10 | medium |
| | | 10° | 18° | 110° | 50% | 30% | present | 130724-005 | 0/100 | 0 | — |
| | | | | | | | absent | 130724-006 | 1/100 | 1 | large |
| | | | | | | 40% | present | 130724-007 | 5/15 | 34 | large, medium |
| | | | | | | | absent | 130724-008 | 3/100 | 3 | medium |
| 20G | 0.90 mm × | 10° | 18° | 110° | 35% | 30% | absent | 130724-009 | 7/60 | 12 | large |
| | 42 mm | | | | | 40% | absent | 130724-010 | 0/100 | 0 | — |
| | | | | | 50% | 30% | absent | 130724-011 | 3/100 | 3 | large |
| | | | | | | 40% | absent | 130724-012 | 0/100 | 0 | — |
| 22G | 0.70 mm × | 10° | 18° | 110° | 35% | 30% | absent | 130724-013 | 0/100 | 0 | — |
| | 42 mm | | | | | 40% | absent | 130724-014 | 1/100 | 1 | small |
| | | | | | 50% | 30% | absent | 130724-015 | 0/100 | 0 | — |
| | | | | | | 40% | absent | 130724-016 | 3/100 | 3 | small |
| 19G | 1.05 mm × | 10° | 18° | 90° | 35% | 30% | present | 130724-017 | 2/100 | 2 | small |
| | 42 mm | | | | | | absent | 130724-018 | 4/100 | 4 | small |
| | | | | | | 40% | present | 130724-019 | 0/100 | 0 | — |
| | | | | | | | absent | 130724-020 | 1/100 | 1 | small |
| | | 10° | 18° | 90° | 50% | 30% | present | 130724-021 | 0/100 | 0 | — |
| | | | | | | | absent | 130724-022 | 7/75 | 4 | large, medium |
| | | | | | | 40% | present | 130724-023 | 5/60 | 8 | small |
| | | | | | | | absent | 130724-024 | 6/20 | 30 | large |

†large: maximum dimension is 2.0 mm or greater medium: maximum dimension is 0.5 to 2.0 mm small: maximum dimension is 0.5 mm or less

TABLE 3

| | | | | | Blade | Blast Condition | | Result | | | |
|---|---|---|---|---|---|---|---|---|---|---|---|
| | | First | | | Surface | Blast | Nozzle | | | Occurrence | |
| Size | | Angle | Second Angle | Rotation Angle | Ratio | Masking | Shift | Cannula Lot | Result | Rate (%) | Core Piece† |
| 20G | 0.90 mm × | 10° | 18° | 90° | 35% | 30% | absent | 130724-025 | 3/30 | 10 | small |
| | 42 mm | | | | | 40% | absent | 130724-026 | 2/100 | 2 | small |
| | | | | | 50% | 30% | absent | 130724-027 | 0/100 | 0 | — |
| | | | | | | 40% | absent | 130724-028 | 1/100 | 1 | small |
| 22G | 0.70 mm × | 10° | 18° | 90° | 35% | 30% | absent | 130724-029 | 0/100 | 0 | — |
| | 42 mm | | | | | 40% | absent | 130724-030 | 0/100 | 0 | — |
| | | | | | 50% | 30% | absent | 130724-031 | 0/100 | 0 | — |
| | | | | | | 40% | absent | 130724-032 | 0/100 | 0 | — |
| 19G | 1.05 mm × | 10° | 18° | 70° | 35% | 30% | present | 130724-033 | 0/100 | 0 | — |
| | 42 mm | | | | | | absent | 130724-034 | 0/100 | 0 | — |
| | | | | | | 40% | present | 130724-035 | 6/45 | 13 | large |
| | | | | | | | absent | 130724-036 | 3/100 | 3 | large |
| | | 10° | 18° | 70° | 50% | 30% | present | 130724-037 | 0/100 | 0 | — |
| | | | | | | | absent | 130724-038 | 2/90 | 2 | large, small |
| | | | | | | 40% | present | 130724-039 | 3/6 | 50 | large |
| | | | | | | | absent | 130724-040 | 4/10 | 40 | large |
| 20G | 0.90 mm × | 10° | 18° | 70° | 35% | 30% | absent | 130724-041 | 0/100 | 0 | — |
| | 42 mm | | | | | 40% | absent | 130724-042 | 0/100 | 0 | — |
| | | | | | 50% | 30% | absent | 130724-043 | 0/100 | 0 | — |
| | | | | | | 40% | absent | 130724-044 | 0/100 | 0 | — |
| 22G | 0.70 mm × | 10° | 18° | 70° | 35% | 30% | absent | 130724-045 | 0/100 | 0 | — |
| | 42 mm | | | | | 40% | absent | 130724-046 | 0/100 | 0 | — |
| | | | | | 50% | 30% | absent | 130724-047 | 0/100 | 0 | — |
| | | | | | | 40% | absent | 130724-048 | 0/100 | 0 | — |

†large: maximum dimension is 2.0 mm or greater medium: maximum dimension is 0.5 to 2.0 mm small: maximum dimension is 0.5 mm or less

TABLE 4

| | | | | | | | | 1st Lot | | | 2nd Lot | | |
|---|---|---|---|---|---|---|---|---|---|---|---|---|---|
| | | | | | | | | | Puncture | | | Puncture | |
| | | | | | | | | | Resistance | | | Resistance | |
| | | | | | | | | | Test (Average | | | Test (Average | |
| | | | | Blade | | Nozzle | | | Value) | | | Value) | |
| | First | Second | Rotation | Surface | Blast | Shift | Number of | | Blade | | | Blade | |
| Gauge Size | Angle | Angle | Angle | Ratio | Masking | present/ | Blasting | Cannula | Edge | Cannula | Cannula | Edge | Cannula |
| G | ° | ° | ° | % | % | absent | number | Lot | (gf) | (gf) | Lot | (gf) | (gf) |
| 19G × 42 mm | 10 | 18 | 70 | 50 | 30 | present | 4 round | 141006-1 | 60.5 | 22.5 | 141006-3 | 57.1 | 19.8 |
| | | | | 35 | | | trips | 141006-2 | 46.5 | 20.1 | 141006-4 | 46.1 | 18.8 |
| 20G × 42 mm | 10 | 18 | 70 | 50 | 30 | present | 4 round | 141006-7 | 53.2 | 17.7 | 141006-9 | 50.4 | 16.8 |
| | | | | 35 | | | trips | 141006-8 | 42.4 | 16.4 | 141006- | 46.0 | 17.8 |
| | | | | | | | | | | | 10 | | |
| 22G × 42 mm | 10 | 18 | 70 | 50 | 30 | present | 4 round | 141006- | 48.5 | 14.9 | 141006- | 47.2 | 14.8 |
| | | | | | | | trips | 13 | | | 15 | | |
| | | | | 35 | | | | 141006- | 40.6 | 14.8 | 141006- | 42.5 | 14.0 |
| | | | | | | | | 14 | | | 16 | | |

TABLE 5

| | | | | | | | | 3rd Lot | | |
|---|---|---|---|---|---|---|---|---|---|---|
| | | | | Blade | | | | | Puncture Resistance | |
| | First | Second | Rotation | Surface | Blast | | Number of | | Test (Average Value) | |
| Gauge Size | Angle | Angle | Angle | Ratio | Masking | Nozzle Shift | Blasting | | Blade | |
| G | ° | ° | ° | % | % | present/absent | number | Cannula Lot | Edge (gf) | Cannula (gf) |
| 19G × 42 mm | 10 | 18 | 70 | 50 | 30 | present | 4 round | 141006-5 | 56.7 | 19.2 |
| | | | | 35 | | | trips | 141006-6 | 48.6 | 19.1 |
| 20G × 42 mm | 10 | 18 | 70 | 50 | 30 | present | 4 round | 141006-11 | 49.4 | 17.2 |
| | | | | 35 | | | trips | 141006-12 | 48.2 | 18.5 |
| 22G × 42 mm | 10 | 18 | 70 | 50 | 30 | present | 4 round | 141006-17 | 48.2 | 15.0 |
| | | | | 35 | | | trips | 141006-18 | 43.4 | 18.1 |

From the tables shown above, the frequency of occurrence of coring tended to increase as the gauge size became thicker.

As shown in Table 2 and Table 3, the frequency of coring tended to increase as the gauge size became thicker. Additionally, the frequency of coring tended to decrease when nozzle shift was present. The frequency of coring tended to decrease when a needle had a blast masking ratio of 30%. Further, a needle having a rotation angle of 70% showed decreased coring.

In some puncture needles, there were few variations and tendency difference between the manufacturing lots. However, as to the frequency of occurrence of coring in accordance with each factor, the occurrence of coring tended to decrease under the treatment conditions that the blast masking ratio was 30%, the blade surface ratio was 50% and the rotation angle was 70°.

As will be understood by those skilled in the art who have the benefit of the instant disclosure, other equivalent or alternative puncture needles, methods, and systems can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. In addition, the size of a device and/or system may be scaled up (e.g., to be used for adult subjects) or down (e.g., to be used for juvenile subjects) to suit the needs and/or desires of a practitioner. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Where open terms such as “having” or “comprising” are used, one of ordinary skill in the art having the benefit of the instant disclosure will appreciate that the disclosed features or steps optionally may be combined with additional features or steps. Such option may not be exercised and, indeed, in some embodiments, disclosed systems, compositions, apparatuses, and/or methods may exclude any other features or steps beyond those disclosed herein. Elements, compositions, devices, systems, methods, and method steps not recited may be included or excluded as desired or required. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value+/−about 10%, depicted value+/−about 50%, depicted value+/−about 100%) and/or a range endpoint. With respect to the former, a value of 50 depicted in an example, table, and/or drawing may form the basis of a range of, for example, about 45 to about 55, about 25 to about 100, and/or about 0 to about 100. Disclosed percentages are weight percentages except where indicated otherwise.

All or a portion of a device and/or system for a puncture needle may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

What is claimed is:

1. A puncture needle comprising a cannula provided with a lumen, wherein
said cannula has a blade surface,
said blade surface has:
a sharp region located on a tip side of said blade surface and provided with a sharp edge on inner and outer circumferences of said blade surface; and
a blunt region located on a base end side of said blade surface and provided with a blunt edge on the inner and outer circumferences of said blade surface, and
a tip of said blade surface is provided with a thick portion formed by a boundary between two surfaces, and said boundary forms an edge.

2. The puncture needle according to claim 1, wherein
said blunt region is formed to extend over a half or more of a height of said cannula.

3. The puncture needle according to claim 2, wherein
said blade surface has a first surface located on the base end side and a second surface located on the tip side, and a side surface ridge line which is a boundary between said first surface and said second surface is located in said blunt region.

4. The puncture needle according to claim 3, wherein
a length of said sharp region is less than or equal to 40% of a length of said blade surface.

5. The puncture needle according to claim 3, wherein
the blade surface of the second surface has a shape having a curvature that becomes gradually smaller from the tip side toward the base end side.

6. The puncture needle according to claim 3, wherein
said second surface has a right-side second surface and a left-side second surface, and an angle formed by said right-side second surface with respect to said left-side second surface is greater than or equal to 66° and less than or equal to 114°.

7. The puncture needle according to claim 6, wherein
a ratio of a length of said second surface to a length of said blade surface is greater than or equal to 35% and less than or equal to 50%.

8. The puncture needle according to claim 7, wherein
in a range where a length from the tip of said blade surface is greater than or equal to 30% and less than or equal to 40% of the length of said blade surface, the sharp edge is formed on the inner and outer circumferences of said blade surface.

9. The puncture needle according to claim 8, wherein
the angle formed by said right-side second surface with respect to said left-side second surface is greater than or equal to 66° and less than or equal to 94°.

10. The puncture needle according to claim 9, wherein
the length of said second surface is greater than or equal to 46% and less than or equal to 54% of the length of said blade surface.

11. The puncture needle according to claim 10, wherein
the angle formed by said right-side second surface with respect to said left-side second surface is greater than or equal to 66° and less than or equal to 74°.

12. The puncture needle according to claim 11, wherein
in a range where the length from the tip of said blade surface is greater than or equal to 26% and less than or equal to 34% of the length of said blade surface, the sharp edge is formed on the inner and outer circumferences of said blade surface.

13. The puncture needle according to claim 12, wherein
said blunt region is formed by blast treatment, and an area of the surface subjected to blast treatment is larger on the base end side than on the tip side in said first surface, and larger on the base end side than on the tip side in said second surface.

14. The puncture needle according to claim 13, wherein
said cannula exposed from a hub has a linear shape.

15. A puncture needle comprising a needle wall defining a central longitudinal axis, the central longitudinal axis positioned within a first plane and within a second plane that perpendicularly intersects the first plane, the needle wall comprising:
an inner needle wall surface;
an outer needle wall surface;
a first zone with a circumferentially contiguous tubular configuration that fully encircles the central longitudinal axis;
a second zone that partially encircles the central longitudinal axis, the second zone comprising:
a first phase that adjoins and extends contiguously from the first zone,
a second phase that adjoins and extends contiguously from the first phase, and
a lateral opening spanning the first and second phases, defining a blade surface consisting of a first portion positioned in the first phase and a second portion positioned in the second phase, and defining an inner blade circumference and an outer blade circumference,
wherein the first portion of the blade surface is positioned in a third plane, the third plane oblique to the central longitudinal axis, oblique to the first plane, and perpendicular to the second plane,
wherein the second portion of the blade surface is positioned generally in a plane curve, the plane curve oblique to the central longitudinal axis, oblique to the first plane, perpendicular to the second plane, and oblique to the third plane; and
a third zone that only partially encircles the central longitudinal axis and forms a needle tip, the third zone adjoining and extending contiguously from the second phase of the second zone, wherein a section of the needle tip, perpendicular to the longitudinal axis and taken anywhere in the third zone, forms a partial Reuleaux triangle in which the base is a circular segment of radius (Rx) defined by the outer surface and two straight sides, each defined by the blade surface, and together defining an angle α^{tip}, wherein a length of the two straight sides and a circumference of the circular segment generally decrease in successive sections perpendicular to the central longitudinal axis moving toward a distal extent of the needle tip,
wherein the first portion of the blade surface is roughened.

16. The puncture needle according to claim 15, wherein the angle α^{tip} may be from about 70° to about 110°.

17. The puncture needle according to claim 15, wherein the angle α^{tip} may be constant across the third zone.

18. The puncture needle according to claim 15, wherein a section of the second phase of the second zone, perpendicular to the longitudinal axis and taken anywhere in the second phase of the second zone, forms an annular sector defined by an outer needle wall surface sector, an inner needle wall surface sector, a left blade surface, and a right blade surface, lines extending from the left and right blade surfaces intersecting to form an angle θ.

19. The puncture needle according to claim 18, wherein the angle θ may be from about 70° to about 110°.

20. The puncture needle according to claim 18, wherein the angle θ may be constant across the third zone.

21. A puncture needle comprising a cannula 6 provided with a lumen 3, the cannula 6 comprising a puncture portion 4, the puncture portion 4 comprising:
a first surface 10, across left- and right-sides, distal to a tip 31 of the puncture portion 4;
a left-side second surface 22 proximal to the tip 31 of the puncture portion 4; and
a right-side second surface 21 proximal to the tip 31 of the puncture portion 4,
wherein the first surface 10 connects to the left-side second surface 22 via a left ridge 33 and connects to the right-side second surface 21 via a right ridge 34,
wherein the left-side second surface 22 and the right-side second surface 21 intersect at a sharp edge 37 extending from the tip 31 of the puncture portion to a tip-end of the lumen 3, and
wherein, from the perspective of a plane defined by the sharp edge 37 and a longitudinal direction 1a of the cannula 6, both inner and outer circumferences of the left-side second surface 22 are recessed and curved, and both inner and outer circumferences of the right-side second surface 21 are recessed and curved,
wherein the first surface 10 is blast-treated for reducing coring, and wherein part of the left-side second surface 22 and of the right-side second surface 21 that extends from the tip 31 is not blast-treated.

22. The puncture needle of claim 21, wherein an inclination angle of the sharp edge 37 (α), with respect to the longitudinal direction 1a, is greater than an inclination angle of the first surface 10 (β), with respect to the longitudinal direction 1a.

23. The puncture needle of claim 22, wherein the inclination angle of the sharp edge 37 (α) is greater than an inclination angle of the inner circumference of the left-side second surface 22, at the tip-end of the lumen 3, with respect to the longitudinal direction 1a.

24. The puncture needle of claim 22, wherein the inclination angle of the first surface 10 (β) is smaller, than an inclination angle of the inner circumference of the left-side second surface 22 at the left ridge 33, with respect to the longitudinal direction 1a.

25. The puncture needle of claim 21, wherein, from the perspective of a cross-sectional plane perpendicular to the longitudinal direction 1a, the left-side second surface 22 defines a first line and the right-side second surface 21 defines a second line, and wherein an angle between the first and second lines is no less than about 70° and no greater than about 110°.

26. The puncture needle of claim 21, wherein a length of the first surface 10 along the longitudinal direction 1a is between about 46% and about 54% of a length of the puncture portion 4 along the longitudinal direction 1a.

28. The puncture needle of claim 21, wherein about 70% of the puncture portion 4, by length along the longitudinal direction 1a, is blast-treated, and wherein the blast-treated part is distal to the tip 31.

29. A puncture needle comprising a cannula 6, the cannula 6 comprising a puncture portion 4 for insertion into a septum, the puncture portion 4 comprising a blade surface 2 inclined with respect to a longitudinal direction of the cannula 6, the blade surface 2 comprising:
a first surface 10 proximal to a base-end of the blade surface 2;
a left-side second surface 21 proximal to a tip of the blade surface 2; and
a right-side second surface 22 parallel to the left-side second surface 21 with respect to the longitudinal direction of the cannula 6,
wherein part of the blade surface 2 distal to a tip of the puncture portion is blast-treated, and wherein part of the blade surface 2 extending from the tip of the puncture portion 4 is not blast-treated, and
wherein the first surface 10 intersects both the left-side second surface 21 and the right-side second surface 21 at least one common angle less than 180°, wherein at least part of the first surface 10 is blast-treated for reducing coring, and wherein parts of the left-second surface 21 and the right-side second surface 22 extending from the tip of the blade surface 2 are not blast-treated.

30. A puncture needle comprising a cannula 6 provided with a lumen 3, the cannula 6 comprising a blade surface 2 with an inner edge circumference 35 and an outer circumference 36, the blade surface 2 comprising:
a blunt region 39a located on a base-end side of the blade surface 2, the blunt region 39a comprising a first blunt edge on the inner edge circumference 35 of the blade surface 2 and a second blunt edge on the outer circumference 36 of the blade surface 2; and
a sharp region 39b located on a tip side of the blade surface 2, the sharp region 39b comprising a first sharp edge on the inner edge circumference 35 of the blade surface 2 and a second sharp edge on the outer circumference 36 of the blade surface 2,
wherein the sharp region 39b further comprises a left-side surface 21 and a right-side surface 22, wherein the left-side surface 21 and the right-side surface 22 intersect, with an angle between about 66° to about 114°, at a straight boundary line 37 extending from a tip end 31 of the outer circumference 36 to a tip end of the inner edge circumference 35, wherein an inclination angle of the straight boundary line 37 (α³⁷), with respect to a longitudinal direction 1a of the cannula 6, is greater than (1) an inclination angle of the outer circumference 36 at the tip end 31 of the outer circumference 36 and (2) an inclination angle of the inner edge circumference 35 at the tip end of the inner edge circumference 35, wherein the left-side surface 21 and the right-side surface 22 have a recessed, curved profile in a side view that is parallel to a plane defined by the straight boundary line 37 and the longitudinal direction of the cannula 6,
wherein the blunt region 39a intersects the left-side surface 21 at a first ridge 33 and intersects the right-side second surface 22 at a second ridge 34, wherein a length of the sharp region 39b is between about 46% and about 54% of a length of the blade surface 2 along the longitudinal direction 1a of the cannula 6, wherein inclination angles of the first and second sharp edges of the sharp region 39b are greater than inclination angles of the first and second blunt edge of the blunt region 39a, respectively, with respect to the longitudinal direction 1a of the cannula 6 at the first and second ridges 33 and 34,
wherein at least part of the blunt region 39a distal to the tip end 31 of the outer circumference 36 is blast-treated for reducing coring, and wherein part of the sharp region 39b extending from the tip end 31 of the outer circumference 36 is not blast-treated.
